SEQUENCE LISTING (1 sequence)

SEQ ID NO: 1 - protein, 149 aa, Homo sapiens
MRKTRWGWMVSRAATKTKYKGTDVKCDYTKASSKAWRDGMKTACTRSKNSHVVGRDYHDHGRVRMVNVDSGYCVYKHMDRRVVTKGSGTGSNNSTNVYKTTTKACYTSRTVTAKSTADVSTDSNTNVTDRVVNVAGGSKSVSVAVTRSV

(19) United States
(12) Patent Application Publication
(10) Pub. No.: US 2018/0318379 A1
(43) Pub. Date: Nov. 8, 2018

(54) INHIBITION OF TRIGGERING RECEPTOR EXPRESSED ON MYELOID CELLS 1 (TREM1) TO TREAT CENTRAL NERVOUS SYSTEM DISORDERS

(71) Applicant: The Board of Trustees of the Leland Stanford Junior University, Stanford, CA (US)

(72) Inventor: Katrin Andreasson, Stanford, CA (US)

(21) Appl. No.: 15/967,260

(22) Filed: Apr. 30, 2018

Related U.S. Application Data
(60) Provisional application No. 62/492,645, filed on May 1, 2017.

(51) Int. Cl. A61K 38/03; A61P 25/28; A61K 45/06; A61P 25/16

(52) U.S. Cl. CPC A61K 38/03; A61P 25/16; A61K 45/06; A61P 25/28

(57) ABSTRACT

Aspects of the present invention include treating a subject having an acute of chronic central nervous system disorder, such as a brain disorder or spinal cord disorder, by administering an agent that inhibits TREM1 activity and/or expression.

### CROSS-REFERENCE

Pursuant to 35 U.S.C. § 119 (e), this application claims priority to the filing date of the U.S. Provisional Patent Application No. 62/492,645 filed May 1, 2017, which application is incorporated herein by reference in its entirety.

### GOVERNMENT RIGHTS

This invention was made with Government support under contract TR001085 awarded by the National Institutes of Health. The Government has certain rights in the invention.

### BACKGROUND OF THE INVENTION

There is a critical need for new preventive and therapeutic agents for treating a multitude of brain disorders. Recent studies have indicated that inhibiting maladaptive neuroinflammation in brain disorders could provide clinical benefits. For example, genome-wide association studies (GWAS) and systems biology approaches in humans demonstrate that the neuroinflammatory response is dominant in increasing risk of and/or the severity of Alzheimer's disease.

### SUMMARY

The present disclosure shows the role of TREM1 signaling in promoting a maladaptive immune response. Specifically, increasing TREM1 activity and declining TREM2 activity favors a maladaptive immune response that worsens neuronal and synaptic outcomes. TREM1 inhibition is thus a good strategy for treating central nervous system disorders (e.g., brain disorders, spinal cord disorders) in a subject, where central nervous system disorders (e.g., brain disorders) are generally associated with dysregulated immune responses and maladaptive myeloid function that can lead to neuronal and circuit injury. TREM1 inhibition can be viewed as an instrument to enhance opposing signaling activity of TREM2, as they both signal through the same adapter protein, DAP12. Thus, reducing TREM1 activity relative to TREM2 activity in a subject can treat a central nervous system (e.g., brain) disorder in a subject.

### BRIEF DESCRIPTION OF THE DRAWINGS

FIG. 1. TREM1/TREM2 balance: beneficial vs maladaptive microglial responses. (1) TREM1 functionally opposes TREM2: TREM1 amplifies the pro-inflammatory response, TREM2 is anti-inflammatory and pro-phagocytic. (2) Both TREM1 and TREM2 signal through same adapter protein: DAP12. Microglial/myeloid immune response genes are the most highly overrepresented in AD in systems biology studies, and DAP12 is a central regulator of AD immune responses. (3) Human genetics: GWAS: TREM2 SNP increases risk of AD; Intronic variant in promoter region of TREM1 is associated with increased CERAD score and severity of cognitive decline.

FIG. 2. TREM1/TREM2 both signal through DAP12 to mediate opposite inflammatory responses. TREM1 signaling promotes/enhances: ROS and oxidative stress, pro-inflammatory cytokine expression, resulting in amplification of maladaptive immune responses. TREM2 signaling promotes/enhances: anti-inflammatory responses, and increased phagocytosis/clearance of toxic substances.

FIG. 3. TREM1 mediates maladaptive inflammatory responses. (1) TREM1 functions as an amplifier of toxic inflammation; must have co-activation of TLRs, NLRs, other innate immune receptors. (2) TREM1 is only expressed on myeloid-lineage cells. (3) Extensive data in peripheral inflammatory systems showing amplifier function using TREM1 KO and TREM1 peptide inhibitors in vivo (intestinal colitis, sepsis models).

FIG. 4. Reciprocal expression of TREM1 vs TREM2 mRNA in BV2 microglial cells in response to Aβ42 oligomers. Quantitative PCR was carried out on BV2 microglial cells stimulated with different concentrations of Aβ42 oligomers, and shows an increase in TREM1 mRNA and a converse decrease in TREM2 mRNA. ANOVA values are shown for TREM1 (red) and TREM2 (green).

FIG. 5. Reciprocal expression of TREM1 vs TREM2 mRNA in primary myeloid cells in response to Aβ42 oligomers. Mouse primary microglia and mouse primary peritoneal macrophages were cultured and stimulated with Aβ42 oligomers (10 μM and 5 μM, respectively) and mRNA was quantified using qRTPCR. Similar to BV2 microglial cells, TREM1 expression increases (t-test, **p<0.01; *p<0.05) and TREM2 expression decreases.

FIG. 6. Surface expression TREM1 protein on BV2 microglial cells in response to LPS and Aβ42 oligomers. A time course of surface expression was assayed using FACS for BV2 cells stimulated with either LPS or Aβ42 oligomers (0.5 and 5 μM) out to 20 hours. Primary macrophages and neutrophils were used as negative and positive controls, respectively.

FIG. 7. TREM1 expression increases in APP-PS1 mice specifically in Iba1+ microglia. APPSwe-PS1ΔE9 mice were aged to 9 mo, and immunocytochemistry was carried out to visualize TREM1, Iba1 (a microglial marker), Aβ42 (using the antibody 6E10) and DAPI to visualize nuclei. (A) in areas distant from amyloid plaques, TREM1 is expressed in Iba1 positive microglia in APP-PS1 brain, but not in non-transgenic brain (not shown). (B) In areas of amyloid plaque deposition, TREM1 is expressed in microglia as well (arrows). (C) TREM1 increases in APP-PS1 mice in parallel to increases in Iba1, and correlate tightly to Iba1 levels.

FIG. 8. TREM1 protein increases in human superior temporal cortex in AD. Quantitative Western analysis of lysates of human superior temporal cortex from post-mortem tissues of subjects (control, MCI, and AD; n=3 per group) demonstrates an increase in TREM1 expression with progression to AD (t-test, *p<0.05).

FIG. 9. TREM1 immunoreactivity increases in microglia in human superior temporal cortex in AD. Immunostaining of TREM1 in control and AD superior temporal lobe demonstrates microglial staining with TREM1 (brown). Also shown is AB deposition, with small amyloid plaque devoid of microglia in control brain, and a large amyloid plaque surrounded by activated TREM1 positive microglia in the AD section.

FIG. 10. TREM1/TREM2 balance: relevance to CNS as well as peripheral diseases. The overall neuroinflammatory response will reflect the balance between TREM1 and TREM2 activities. If TREM1 is dominant, a maladaptive immune response will ensue. If TREM1 is suppressed, TREM2 will elicit a beneficial homeostatic immune response in brain. CNS diseases: Acute: TBI, stroke, spinal cord injury; Subacute: epilepsy, CTE, pain; Chronic: AD, PD, and other neurodegenerative diseases. Peripheral inflammatory diseases: Arthritis; Autoimmune disorders: RA, IBD, colitis; Pancreatitis; Gout; Cancer.

FIG. 11. A timeline showing windows of intervention after an ischemic event. Post-stroke inflammatory response provides an attractive and long window for intervention.

FIG. 12. A representation of the dynamics of immune present at the indicated days after stroke (or the temporal dynamics of immune cell subsets). Stroke was modeled in C576/6 3 mo male mice using 45 minutes of middle cerebral artery occlusion (MCAo) followed by reperfusion (RP).

FIG. 13. FACS quantification of TREM1 surface expression in brain myeloid cells at 48 hours after MCAo-RP. TREM1 surface expression is increased in infiltrating macrophages and to a lesser extent in infiltrating neutrophils 48 hours after MCAo-RP.

FIG. 14. FACS quantification of TREM1 and TREM2 expressing macrophages and neutrophils at 2 days and 6 days after MCAo-RP during the post-stroke inflammatory response. TREM1 expression in myeloid cells (macrophages and neutrophils) is initially high and then decreases whereas TREM2 expression is initially low and then increases.

FIG. 15. Plots showing reciprocal expression of TREM1 and TREM2 mRNA in RAW mouse macrophage cell line in response to LPS: TREM1 increases and TREM2 decreases. ANOVA p values are shown for TREM1 (red) and TREM2 (green).

FIG. 16. Neuroscore plot (panel A), FACS plots (panel B), and myeloid cell counts (panel C) showing that TREM1 peptide decoy LP17 (Gibot S, Kolopp-Sarda M N, Bene M C, Bollaert P E, Lozniewski A, Mory F, et al. A soluble form of the triggering receptor expressed on myeloid cells-1 modulates the inflammatory response in murine sepsis. J Exp Med. 2004; 200(11):1419-26) reduces infiltration of macrophages after MCAo-RP stroke.

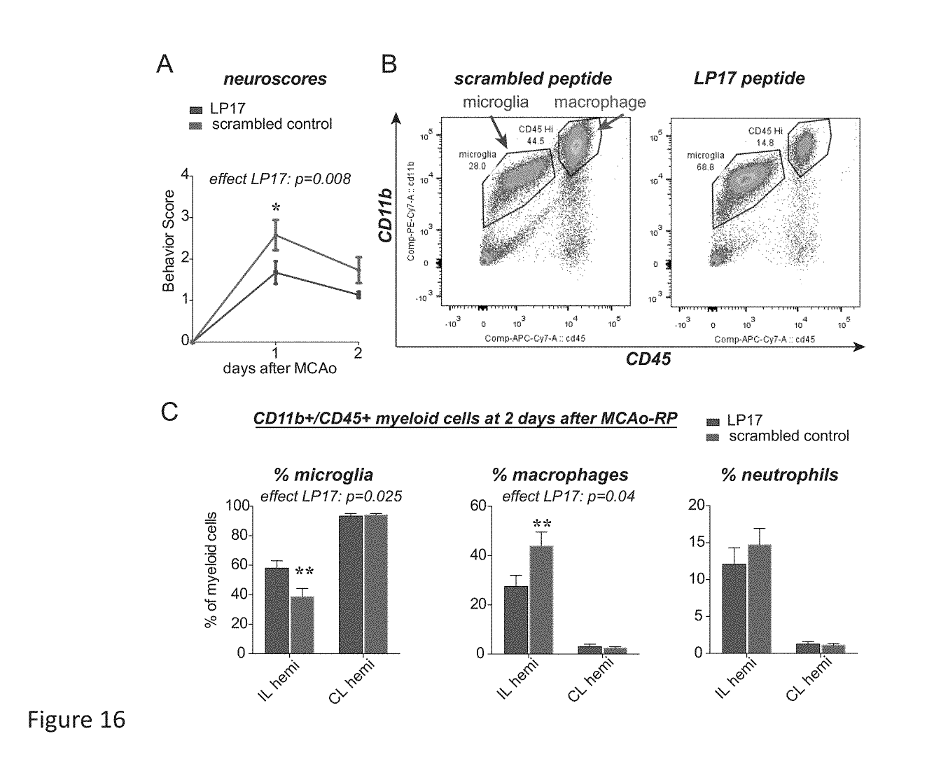

### DEFINITIONS

In the description that follows, a number of terms conventionally used in the field of cell culture are utilized. In order to provide a clear and consistent understanding of the specification and claims, and the scope to be given to such terms, the following definitions are provided.

The terms “TREM1 inhibitory agent” and the like are agents that decrease TREM1 (NCBI human gene ID: 54210; NCBI mouse gene ID: 58217) activity in a cell. Such agents can inhibit the signaling activity of the TREM1 receptor, reduce its expression, or both. The reduction in TREM1 activity can be relative to TREM2 activity, such that an inhibitory agent that increases TREM2 activity as compared to TREM1 activity can be a TREM1 inhibitory agent.

TREM1 inhibitory agents can take any convenient form, including polypeptides or proteins (e.g., inhibitory peptides, antibodies or binding fragments thereof, and the like), nucleic acids (e.g., RNAi agents), small molecules, etc. No limitation in this regard is intended.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and 0-phosphoserine. An amino acid analog refers to a compound that has the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

The term “central nervous system disorder” includes an acute or chronic disorder of the brain and/or spinal cord in a subject that exhibits a maladaptive immune or inflammatory response. The term “brain disorder” includes an acute or chronic brain disorder in a subject that exhibits a maladaptive immune or inflammatory response. A maladaptive immune response is one where the immune response either perpetuates the underlying injury or worsens it. Non limiting examples of central nervous system and brain disorders of interest include: stroke, head trauma, spinal cord injury, seizures, encephalitis (acute disorders); and Alzheimer's disease, Parkinson's disease, fronto-temporal dementia, amyotrophic lateral sclerosis, Huntington's disease, multiple sclerosis, pain, depression, PTSD, and post-stroke inflammation, post-traumatic inflammation, and chronic fatigue syndrome (chronic disorders). In some cases the brain disorder of a subject method is stroke. It is to be understood that when the term “brain disorder” is used throughout this disclosure, e.g., in the context of a subject method, the same method and/or composition can be used to treat a “central nervous system disorder,” which term encompasses the term “brain disorder.”

Alzheimer's disease (AD). Alzheimer's disease is a progressive, inexorable loss of cognitive function associated with an excessive number of senile plaques in the cerebral cortex and subcortical gray matter, which also contains b-amyloid and neurofibrillary tangles consisting of tau protein. The common form affects persons >60 yr old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, synapses, and ultimately neurons are lost within the cerebral cortex, hippocampus, and subcortical structures (including selective cell loss in the nucleus basalis of Meynert), locus caeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

Parkinson's Disease. Parkinson's Disease (PD) is an idiopathic, slowly progressive, degenerative CNS disorder characterized by slow and decreased movement, muscular rigidity, resting tremor, and postural instability. Originally considered primarily a motor disorder, PD is now recognized to also affect cognition, behavior, sleep, autonomic function, and sensory function. The most common cognitive impairments include an impairment in attention and concentration, working memory, executive function, producing language, and visuospatial function.

In primary Parkinson's disease, the pigmented neurons of the substantia nigra, locus caeruleus, and other brain stem dopaminergic cell groups are lost. The cause is not known. The loss of substantia nigra neurons, which project to the caudate nucleus and putamen, results in depletion of the neurotransmitter dopamine in these areas. Onset is generally after age 40, with increasing incidence in older age groups.

Secondary parkinsonis results from loss of or interference with the action of dopamine in the basal ganglia due to other idiopathic degenerative diseases, drugs, or exogenous toxins. The most common cause of secondary parkinsonism is ingestion of antipsychotic drugs or reserpine, which produce parkinsonism by blocking dopamine receptors. Less common causes include carbon monoxide or manganese poisoning, hydrocephalus, structural lesions (tumors, infarcts affecting the midbrain or basal ganglia), subdural hematoma, and degenerative disorders, including striatonigral degeneration.

Frontotemporal dementia. Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. Symptoms are classified into three groups based on the functions of the frontal and temporal lobes affected: Behavioural variant FTD (bvFTD), with symptoms include lethargy and aspontaneity on the one hand, and disinhibition on the other; progressive nonfluent aphasia (PNFA), in which a breakdown in speech fluency due to articulation difficulty, phonological and/or syntactic errors is observed but word comprehension is preserved; and semantic dementia (SD), in which patients remain fluent with normal phonology and syntax but have increasing difficulty with naming and word comprehension. Other cognitive symptoms common to all FTD patients include an impairment in executive function and ability to focus. Other cognitive abilities, including perception, spatial skills, memory and praxis typically remain intact. FTD can be diagnosed by observation of reveal frontal lobe and/or anterior temporal lobe atrophy in structural MRI scans.

A number of forms of FTD exist, any of which may be treated or prevented using the subject methods and compositions. For example, one form of frontotemporal dementia is Semantic Dementia (SD). SD is characterized by a loss of semantic memory in both the verbal and non-verbal domains. SD patients often present with the complaint of word-finding difficulties. Clinical signs include fluent aphasia, anomia, impaired comprehension of word meaning, and associative visual agnosia (the inability to match semantically related pictures or objects). As the disease progresses, behavioral and personality changes are often seen similar to those seen in frontotemporal dementia although cases have been described of ‘pure’ semantic dementia with few late behavioral symptoms. Structural MRI imaging shows a characteristic pattern of atrophy in the temporal lobes (predominantly on the left), with inferior greater than superior involvement and anterior temporal lobe atrophy greater than posterior.

As another example, another form of frontotemporal dementia is Pick's disease (PiD, also PcD). A defining characteristic of the disease is build-up of tau proteins in neurons, accumulating into silver-staining, spherical aggregations known as “Pick bodies”. Symptoms include loss of speech (aphasia) and dementia. Patients with orbitofrontal dysfunction can become aggressive and socially inappropriate. They may steal or demonstrate obsessive or repetitive stereotyped behaviors. Patients with dorsomedial or dorsolateral frontal dysfunction may demonstrate a lack of concern, apathy, or decreased spontaneity. Patients can demonstrate an absence of self-monitoring, abnormal self-awareness, and an inability to appreciate meaning. Patients with gray matter loss in the bilateral posterolateral orbitofrontal cortex and right anterior insula may demonstrate changes in eating behaviors, such as a pathologic sweet tooth. Patients with more focal gray matter loss in the anterolateral orbitofrontal cortex may develop hyperphagia. While some of the symptoms can initially be alleviated, the disease progresses and patients often die within two to ten years.

Huntington's disease. Huntington's disease (HD) is a hereditary progressive neurodegenerative disorder characterized by the development of emotional, behavioral, and psychiatric abnormalities; loss of intellectual or cognitive functioning; and movement abnormalities (motor disturbances). The classic signs of HD include the development of chorea—involuntary, rapid, irregular, jerky movements that may affect the face, arms, legs, or trunk—as well as cognitive decline including the gradual loss of thought processing and acquired intellectual abilities. There may be impairment of memory, abstract thinking, and judgment; improper perceptions of time, place, or identity (disorientation); increased agitation; and personality changes (personality disintegration). Although symptoms typically become evident during the fourth or fifth decades of life, the age at onset is variable and ranges from early childhood to late adulthood (e.g., 70s or 80s).

HD is transmitted within families as an autosomal dominant trait. The disorder occurs as the result of abnormally long sequences or “repeats” of coded instructions within a gene on chromosome 4 (4p16.3). The progressive loss of nervous system function associated with HD results from loss of neurons in certain areas of the brain, including the basal ganglia and cerebral cortex.

Amyotrophic lateral sclerosis. Amyotrophic lateral sclerosis (ALS) is a rapidly progressive, invariably fatal neurological disease that attacks motor neurons. Muscular weakness and atrophy and signs of anterior horn cell dysfunction are initially noted most often in the hands and less often in the feet. The site of onset is random, and progression is asymmetric. Cramps are common and may precede weakness. Rarely, a patient survives 30 years; 50% die within 3 years of onset, 20% live 5 years, and 10% live 10 years. Diagnostic features include onset during middle or late adult life and progressive, generalized motor involvement without sensory abnormalities. Nerve conduction velocities are normal until late in the disease. Recent studies have documented the presentation of cognitive impairments as well, particularly a reduction in immediate verbal memory, visual memory, language, and executive function.

A decrease in cell body area, number of synapses and total synaptic length has been reported in even normal-appearing neurons of the ALS patients. It has been suggested that when the plasticity of the active zone reaches its limit, a continuing loss of synapses can lead to functional impairment. Promoting the formation or new synapses or preventing synapse loss may maintain neuron function in these patients.

Multiple Sclerosis. Multiple Sclerosis (MS) is characterized by various symptoms and signs of CNS dysfunction, with remissions and recurring exacerbations. The most common presenting symptoms are paresthesias in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. Common cognitive impairments include impairments in memory (acquiring, retaining, and retrieving new information), attention and concentration (particularly divided attention), information processing, executive functions, visuospatial functions, and verbal fluency. Common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course is highly varied, unpredictable, and, in most patients, remittent. At first, months or years of remission may separate episodes, especially when the disease begins with retrobulbar optic neuritis. However, some patients have frequent attacks and are rapidly incapacitated; for a few the course can be rapidly progressive.

In general, a “maladaptive immune or inflammatory response” is an immune response in a subject that is amplified in intensity and/or duration and leads to an undesirable symptom in the subject. Maladaptive immune or inflammatory responses can include the over- or under-expression of cytokines, chemokines, interleukins, immnomodulatory cell surface receptors, etc., that leads to intense and/or prolonged recruitment and activation of immune cells in a tissue of a subject. These recruited cells can lead to the dysfunction and/or destruction of cells in the affected tissue, e.g., brain cells and synapses.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a central nervous system disorder (e.g., brain disorder) in a mammal, particularly in a human, and includes: (a) preventing the brain disorder or a symptom of a brain disorder from occurring in a subject which may be predisposed to the brain disorder but has not yet been diagnosed as having it (e.g., including brain disorders that may be associated with or caused by a primary disease); (b) inhibiting the brain disorder, i.e., arresting its development; and (c) relieving the brain disorder, i.e., causing regression of the brain disorder.

Treating may refer to any indicia of success in the treatment or amelioration or prevention of a brain disorder, including any objective or subjective parameter such as abatement; remission; diminishing of symptoms or making the brain disorder condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a brain disorder. The term “therapeutic effect” refers to the reduction, elimination, or prevention of the brain disorder, symptoms of the brain disorder, or side effects of the brain disorder in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a brain disorder, is sufficient to effect treatment for that brain disorder.

### DETAILED DESCRIPTION

As summarized above, the present invention relates to treating a subject having a central nervous system disorder, such as brain or spinal cord disorder, by administering a TREM1 inhibitory agent. Treatment using TREM1 inhibition is applicable to multiple brain disorders, including acute disorders such as stroke, head trauma, spinal cord injury, seizures, encephalitis; chronic brain disorders, including Alzheimer's disease, fronto-temporal dementia, Parkinson's disease, amyotrophic lateral sclerosis, Huntington's disease, multiple sclerosis, pain, depression, PTSD, post-stroke inflammation, post-traumatic inflammation, and chronic fatigue syndrome, which are all chronic conditions where a significant and maladaptive neuroinflammatory response has been demonstrated. In some cases a subject method includes administering a TREM1 inhibitory agent (e.g., a blocking peptide such as LP17) to an individual who has had a stroke. In some cases a subject method includes administering a TREM1 inhibitory agent (e.g., a blocking peptide such as LP17) to an individual who at risk of having a stroke.

Before the present methods and compositions are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

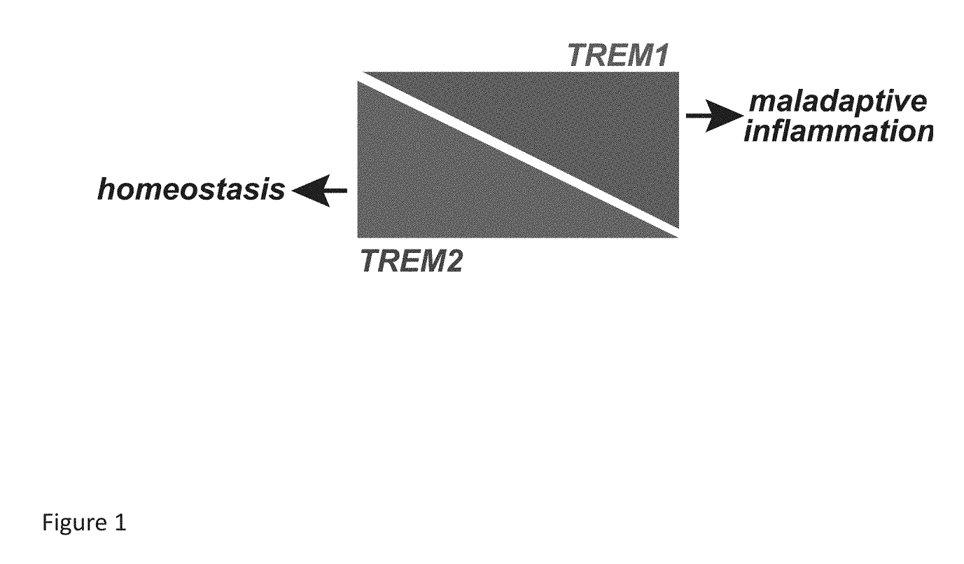

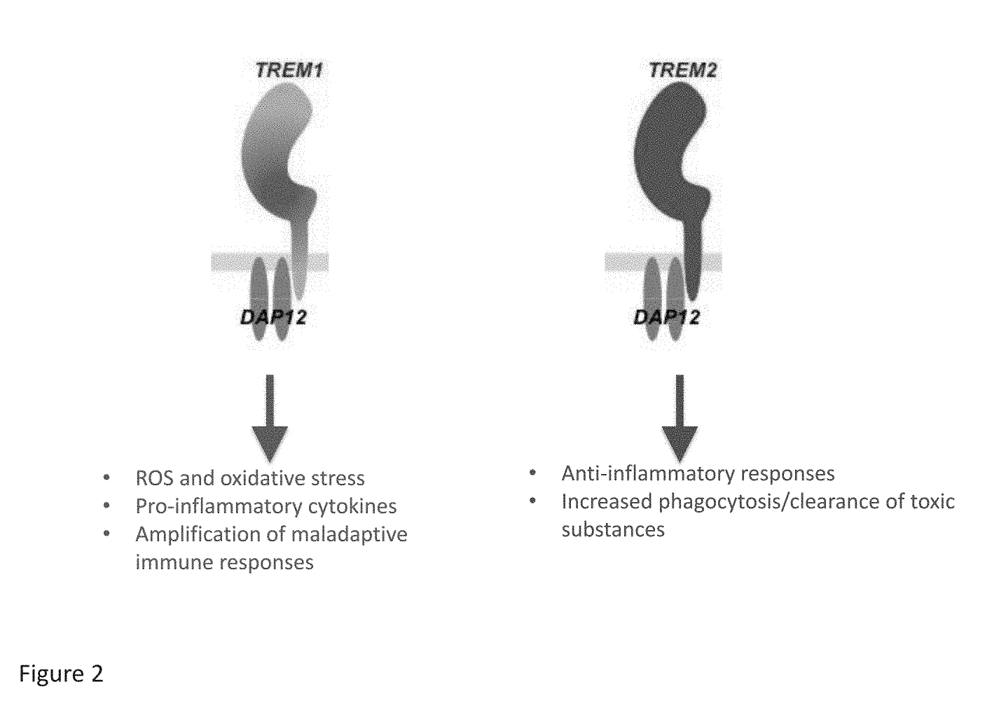

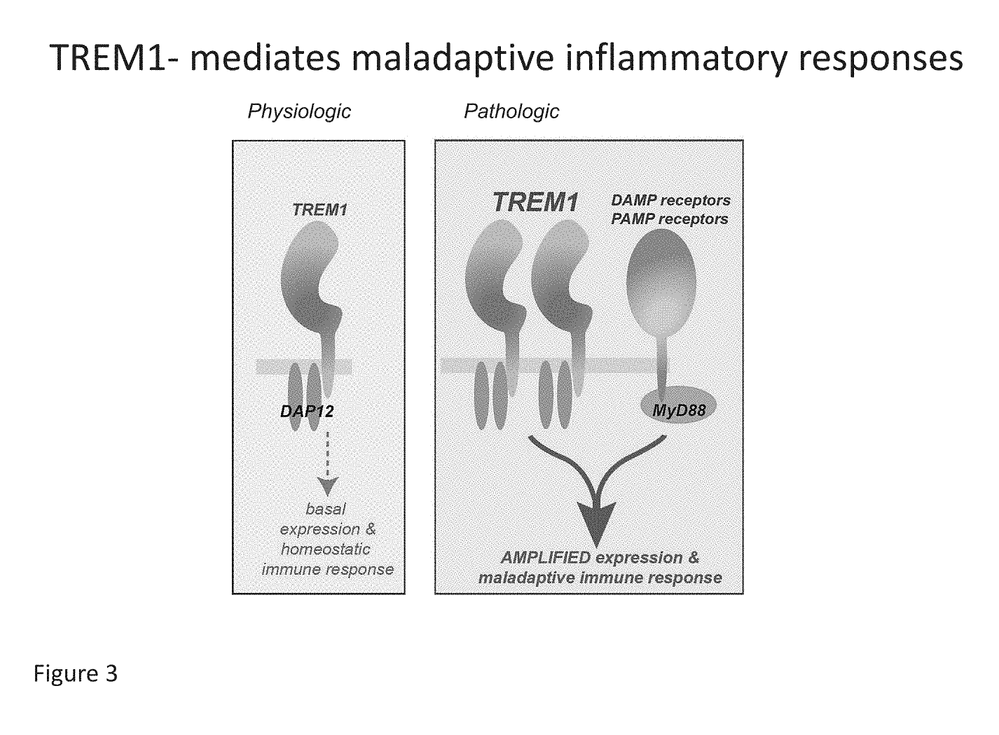

TREM1 is an amplifier of toxic inflammation (see FIGS. 1, 2 and 3). In the brain, such amplification contributes to a maladaptive and injurious immune response that leads to synapse, neuronal, and circuit degeneration. Targeting the immune response in this manner reduces the toxic component of the inflammatory response, leaving the beneficial effects of inflammation (e.g., those mediated by TREM2). In addition, the cell specific expression of TREM1, i.e. by myeloid cells exclusively, and the specific targeting of TREM1 inhibition to myeloid cells (and not neurons, vascular cells, or other non immune cells) addresses this critical problem.

As reviewed above, the terms “TREM1 inhibitory agent” and the like are agents that decrease TREM1 activity in a cell. Such agents can inhibit the signaling activity of the TREM1 receptor, reduce its expression, or both. The reduction in TREM1 activity can be relative to TREM2 activity, such that an inhibitory agent that increases TREM2 activity as compared to TREM1 activity can be a TREM1 inhibitory agent. TREM1 inhibitory agents can take any convenient form, including polypeptides or proteins e.g., antibodies or binding fragments thereof), nucleic acids (e.g., RNAi agents), small molecules, etc. No limitation in this regard is intended.

Depending on the particular embodiments being practiced, a variety of different types of active agents may be employed. In some instances, the agent modulates expression of the RNA and/or protein from the gene, such that it changes the expression of the RNA or protein from the target gene in some manner. In these instances, the agent may change expression of the RNA or protein in a number of different ways. In certain embodiments, the agent is one that reduces, including inhibits, expression of a TREM1 protein. Inhibition of TREM1 protein expression may be accomplished using any convenient means, including use of an agent that inhibits TREM1 protein expression, such as, but not limited to: RNAi agents, antisense agents, agents that interfere with a transcription factor binding to a promoter sequence of the TREM1 gene, or inactivation of the TREM1 gene, e.g., through recombinant techniques, etc.

Reducing (inhibiting) expression and/or function of TREM1 herein refers to reducing protein production (the gene's expression) from the endogenous locus and/or inhibiting the function of the protein that is produced from the endogenous locus (e.g., via genetic mutation resulting in partial or total loss of function allele(s), via small molecule drug, antibody, blocking peptide, and the like). Reducing function of an endogenous gene can be considered to encompass inhibiting/reducing expression of the gene (e.g., by reducing the total amount of protein produced) as well inhibiting/reducing function of a gene product (e.g., protein) encoded/produced by the endogenous gene (e.g., using a small molecule drug, antibody, peptide inhibitor, etc.)—either way, the overall level of function provided by the endogenous locus is reduced/inhibited/blocked.

As would be readily understood by one of ordinary skill in the art, one can reduce expression (protein production) of TREM1 at the DNA, RNA, or protein level. For example, expression can be reduced by reducing the total amount of wild type protein made by the endogenous locus, and this can be accomplished either by changing the nature of the protein produced (e.g., via gene mutation to generate a loss of function allele such as a null allele or an allele that encodes a protein reduced function) or by reducing the overall levels of protein produced without changing the nature of the protein itself.

Reducing (inhibiting) expression and/or function of an endogenous gene (TREM1) can be accomplished using any convenient method and one of ordinary skill in the art will be aware of multiple suitable methods. For example, in order to reduce/inhibit expression, one can reduce protein levels post-translationally; one can block production of protein by blocking/reducing translation of mRNA (e.g., using an RNAi agent such as an shRNA or siRNA that targets the mRNA of an endogenous gene to block translation); one can reduce mRNA levels post-transcriptionally (e.g., using an RNAi agent such as an shRNA or siRNA that targets the mRNA of an endogenous gene for degradation); one can reduce mRNA levels by blocking transcription (e.g., using gene editing tools to either alter a promoter and/or enhancer sequence or to modulate transcription, or by using modified gene editing tools, e.g., CRISPRi, that can modify transcription without cutting the target DNA). Additionally, one can alter the nature of the protein made from an endogenous locus by inducing (e.g., using gene editing technology) a loss of function mutation, which can range from an allele with reduced wild type activity to a dead protein or no protein (e.g., catalytically inactive mutant, a frameshift allele, a gene knockout, etc). Moreover, one can reduce mRNA levels via gene editing methods that result in low net transcript levels (e.g., frameshift mutations can trigger nonsense mediated mRNA decay).

Examples of agents that inhibit expression and/or function of an endogenous gene (TREM1) (see above) include but are not limited to: (a) an RNAi agent such as an shRNA or siRNA that specifically targets TREM1 mRNA; (b) a genome editing agent (e.g., a Zinc finger nuclease, a TALEN, a CRISPR/Cas genome editing agent such as Cas9, Cpf1, CasX, CasY, and the like) that cleaves the target cell's genomic DNA at a locus encoding TREM1—thus inducing a genome editing event (e.g., null allele, partial loss of function allele) at the locus; (c) a modified genome editing agent such as a nuclease dead zinc finger, TALE, or CRISPR/Cas nuclease fused to a transcriptional repressor protein that modulates (e.g. reduces) transcription at the locus encoding TREM1 (see, e.g., Qi et al., Cell. 2013 Feb. 28; 152(5):1173-83′; Gilbert et al, Cell. 2014 Oct. 23; 159(3):647-61; Larson et al., Nat Protoc. 2013 November; 8(11):2180-96); and (d) a small molecule/drug that directly blocks/reduces/inhibits the function of the protein produced by the endogenous locus.

When the agent is a CRISPR/Cas editing agent, the agent can include both the protein and guide RNA component. The guide nucleic acid (e.g., guide RNA) can be introduced into the cell as an RNA or as a DNA encoding the RNA (e.g., encoded by a DNA vector—on a plasmid, virus, and the like). The CRISPR/Cas protein can be introduced into the cell as a protein or as a nucleic acid (mRNA or DNA) encoding the protein. For additional information related to programmable gene editing agents and their guide nucleic acids (e.g., CRISPR/Cas RNa-guided proteins such as Cas9, CasX, CasY, and Cpf1, Zinc finger proteins such as Zinc finger nucleases, TALE proteins such as TALENs, CRISPR/Cas guide RNAs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; Burstein et al., Nature. 2016 Dec. 22—Epub ahead of print; Gao et al., Nat Biotechnol. 2016 July 34(7):768-73; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

As noted above, the transcription level of a TREM1 protein can be regulated by gene silencing using RNAi agents, e.g., double-strand RNA (see e.g., Sharp, Genes and Development (1999) 13: 139-141). RNAi, such as double-stranded RNA interference (dsRNAi) or small interfering RNA (siRNA), has been extensively documented in the nematode C. elegans (Fire, et al, Nature (1998) 391:806-811) and routinely used to “knock down” genes in various systems. RNAi agents may be dsRNA or a transcriptional template of the interfering ribonucleic acid which can be used to produce dsRNA in a cell. In these embodiments, the transcriptional template may be a DNA that encodes the interfering ribonucleic acid. Methods and procedures associated with RNAi are also described in published PCT Application Publication Nos. WO 03/010180 and WO 01/68836, the disclosures of which applications are incorporated herein by reference. dsRNA can be prepared according to any of a number of methods that are known in the art, including in vitro and in vivo methods, as well as by synthetic chemistry approaches. Examples of such methods include, but are not limited to, the methods described by Sadher et al., Biochem. Int. (1987) 14:1015; Bhattacharyya, Nature (1990) 343:484; and U.S. Pat. No. 5,795,715, the disclosures of which are incorporated herein by reference. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference). A number of options can be utilized to deliver the dsRNA into a cell or population of cells such as in a cell culture, tissue, organ or embryo. For instance, RNA can be directly introduced intracellularly. Various physical methods are generally utilized in such instances, such as administration by microinjection (see, e.g., Zernicka-Goetz, et al. Development (1997)124:1133-1137; and Wianny, et al., Chromosoma (1998) 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

In some instances, antisense molecules can be used to down-regulate expression of a TREM1 gene in the cell. The anti-sense reagent may be antisense oligodeoxynucleotides (ODN), particularly synthetic ODN having chemical modifications from native nucleic acids, or nucleic acid constructs that express such anti-sense molecules as RNA. The antisense sequence is complementary to the mRNA of the targeted protein, and inhibits expression of the targeted protein. Antisense molecules inhibit gene expression through various mechanisms, e.g., by reducing the amount of mRNA available for translation, through activation of RNAse H, or steric hindrance. One or a combination of antisense molecules may be administered, where a combination may include multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. Short oligonucleotides, of from 7 to 8 bases in length, can be strong and selective inhibitors of gene expression (see Wagner et al., Nature Biotechnol. (1996) 14:840-844).

A specific region or regions of the endogenous sense strand mRNA sequence are chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra.) Oligonucleotides may be chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O-5′-S-phosphorothioate, 3′-S-5′-O-phosphorothioate, 3′-CH.sub.2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. Nucl. Acids Res. (1995) 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. Appl. Biochem. Biotechnol. (1995) 54:43-56.

In another embodiment, the TREM1 gene is inactivated so that it no longer expresses a functional protein. By inactivated is meant that the gene, e.g., coding sequence and/or regulatory elements thereof, is genetically modified so that it no longer expresses a functional TREM1 protein, e.g., at least with respect to TREM1 aging impairment activity. The alteration or mutation may take a number of different forms, e.g., through deletion of one or more nucleotide residues, through exchange of one or more nucleotide residues, and the like. One means of making such alterations in the coding sequence is by homologous recombination. Methods for generating targeted gene modifications through homologous recombination are known in the art, including those described in: U.S. Pat. Nos. 6,074,853; 5,998,209; 5,998,144; 5,948,653; 5,925,544; 5,830,698; 5,780,296; 5,776,744; 5,721,367; 5,614,396; 5,612,205; the disclosures of which are herein incorporated by reference.

Also of interest in certain embodiments are dominant negative mutants of TREM1 proteins, where expression of such mutants in the cell result in a modulation, e.g., decrease, in TREM1 mediated aging impairment. Dominant negative mutants of TREM1 are mutant proteins that exhibit dominant negative TREM1 activity. As used herein, the term “dominant-negative TREM1 activity” or “dominant negative activity” refers to the inhibition, negation, or diminution of certain particular activities of TREM1. Dominant negative mutations are readily generated for corresponding proteins. These may act by several different mechanisms, including mutations in a substrate-binding domain; mutations in a catalytic domain; mutations in a protein binding domain (e.g., multimer forming, effector, or activating protein binding domains); mutations in cellular localization domain, etc. A mutant polypeptide may interact with wild-type polypeptides (made from the other allele) and form a non-functional multimer. In certain embodiments, the mutant polypeptide will be overproduced. Point mutations are made that have such an effect. In addition, fusion of different polypeptides of various lengths to the terminus of a protein, or deletion of specific domains can yield dominant negative mutants. General strategies are available for making dominant negative mutants (see for example, Herskowitz, Nature (1987) 329:219, and the references cited above). Such techniques are used to create loss of function mutations, which are useful for determining protein function. Methods that are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

In yet other embodiments, the agent is an agent that modulates, e.g., inhibits, TREM1 activity by binding to TREM1 and/or inhibiting binding of TREM1 to a second protein. For example, small molecules that bind to TREM1 and inhibit its activity are of interest. Naturally occurring or synthetic small molecule compounds of interest include numerous chemical classes, such as organic molecules, e.g., small organic compounds having a molecular weight of more than 50 and less than about 2,500 daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below.

In certain embodiments, the administered active agent is a TREM1 specific binding member. In general, useful TREM1 specific binding members exhibit an affinity (Kd) for a target TREM1, such as human TREM1, that is sufficient to provide for the desired reduction in aging associated impairment TREM1 activity. As used herein, the term “affinity” refers to the equilibrium constant for the reversible binding of two agents; “affinity” can be expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of a specific binding member to a target protein can be, for example, from about 100 nanomolar (nM) to about 0.1 nM, from about 100 nM to about 1 picomolar (pM), or from about 100 nM to about 1 femtomolar (fM) or more. The term “binding” refers to a direct association between two molecules, due to, for example, covalent, electrostatic, hydrophobic, and ionic and/or hydrogen-bond interactions, including interactions such as salt bridges and water bridges. In some embodiments, the antibodies bind human TREM1 with nanomolar affinity or picomolar affinity. In some embodiments, the antibodies bind human TREM1 with a Kd of less than about 100 nM, 50 nM, 20 nM, 20 nM, or 1 nM.

Examples of TREM1 specific binding members include TREM1 antibodies and binding fragments thereof. Non-limiting examples of such antibodies include antibodies directed against any epitope of TREM1. Also encompassed are bispecific antibodies, i.e., antibodies in which each of the two binding domains recognizes a different binding epitope. The canonical amino acid sequence of human TREM1 is:

(SEQ ID NO: 01)
MRKTRLWGLL WMLFVSELRA ATKLTEEKYE LKEGQTLDVK
CDYTLEKFAS SQKAWQIIRD GEMPKTLACT ERPSKNSHPV
QVGRIILEDY HDHGLLRVRM VNLQVEDSGL YQCVIYQPPK
EPHMLFDRIR LVVTKGFSGT PGSNENSTQN VYKIPPTTTK
ALCPLYTSPR TVTQAPPKST ADVSTPDSEI NLTNVTDIIR
VPVFNIVILL AGGFLSKSLV FSVLFAVTLR SFVP

Antibody specific binding members that may be employed include full antibodies or immunoglobulins of any isotype, as well as fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. Also encompassed by the term are Fab′, Fv, F(ab′)2, and or other antibody fragments that retain specific binding to antigen, and monoclonal antibodies. An antibody may be monovalent or bivalent.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

Antibodies that may be used in connection with the present disclosure thus can encompass monoclonal antibodies, polyclonal antibodies, bispecific antibodies, Fab antibody fragments, F(ab)2 antibody fragments, Fv antibody fragments (e.g., VH or VL), single chain Fv antibody fragments and dsFv antibody fragments. Furthermore, the antibody molecules may be fully human antibodies, humanized antibodies, or chimeric antibodies. In some embodiments, the antibody molecules are monoclonal, fully human antibodies.

The antibodies that may be used in connection with the present disclosure can include any antibody variable region, mature or unprocessed, linked to any immunoglobulin constant region. If a light chain variable region is linked to a constant region, it can be a kappa chain constant region. If a heavy chain variable region is linked to a constant region, it can be a human gamma 1, gamma 2, gamma 3 or gamma 4 constant region, more preferably, gamma 1, gamma 2 or gamma 4 and even more preferably gamma 1 or gamma 4.

In some embodiments, fully human monoclonal antibodies directed against TREM1 are generated using transgenic mice carrying parts of the human immune system rather than the mouse system.

Minor variations in the amino acid sequences of antibodies or immunoglobulin molecules are encompassed by the present invention, providing that the variations in the amino acid sequence maintain at least 75%, e.g., at least 80%, 90%, 95%, or 99% of the sequence. In particular, conservative amino acid replacements are contemplated. Conservative replacements are those that take place within a family of amino acids that are related in their side chains. Whether an amino acid change results in a functional peptide can readily be determined by assaying the specific activity of the polypeptide derivative. Fragments (or analogs) of antibodies or immunoglobulin molecules, can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Sequence motifs and structural conformations may be used to define structural and functional domains in accordance with the invention.

According to the present invention, TREM1 inhibitory agents can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, pharmaceutical compositions of the present invention include one or more therapeutic entities of the present invention in combination with another therapeutic agent, e.g., another anti-inflammatory agent or additional agent for treating the brain disorder.

In some cases, a TREM1 inhibitory agent is an inhibitory peptide (an antagonist peptide). For example, LP17 (LQVTDSGLYRCVIYHPP) (SEQ ID NO: 1) is one such TREM1 blocking peptide. Thus, in some cases a TREM1 inhibitory agent is LP17. In some cases, a TREM1 inhibitory agent is LR12 (LQEEDTGEYGCV) (SEQ ID NO: 2), which is also a TREM1 blocking peptide. TREM1 blocking antibodies are also known (see, e.g., Brynjolfsson et al., Inflamm Bowel Dis. 2016 August; 22(8):1803-11).

Therapeutic entities of the present invention may be administered as pharmaceutical compositions comprising an active therapeutic agent and other pharmaceutically acceptable excipient(s). The employed form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions of the present invention can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

Methods are provided for treating, reducing or preventing a brain disorder by inhibiting TREM1 activity (or conversely, increasing TREM2 activity). Such methods include administering to a subject in need of treatment a therapeutically a TREM1 inhibitory agent (e.g., an effective amount or an effective dose of a TREM1 inhibitory agent).

As demonstrated in the working examples below, administration of a TREM1 inhibitory agent (e.g., a blocking peptide such as LP17) can cause an increase of CD11b⁺/CD45^{lo} microglia and/or a decrease in CD11b⁺/CD45^{hi} macrophages in the injured region (e.g., after a stroke). Thus, in some cases, a subject method also includes a step of measuring CD11b⁺/CD45^{lo} microglia and/or CD11b⁺/CD45^{hi} macrophages (e.g., in a region of injury in order to assess whether administration of the TREM1 inhibitory agent was successful). For example, a tissue sample such as a biopsy can be used as a sample source for using flow cytometry (e.g., FACS) to count the appropriate cell types. In some cases, a subject method is a method of increasing the number of CD11b⁺/CD45^{hi} macrophages, where the method includes administering a TREM1 inhibitory agent (e.g., a blocking peptide such as LP17).

Effective doses of the therapeutic entity of the present invention, e.g. for the treatment of a brain disorder, vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention can be administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the brain disorder is reduced or terminated, and preferably until the patient shows partial or complete amelioration of symptoms of disease. Thereafter, the patent can be administered a prophylactic regime.

Individuals with a central nervous system disorder (e.g., a brain disorder such as stroke) in some cases include individuals that are about 50 years old or older, e.g., 60 years old or older, 70 years old or older, 80 years old or older, 90 years old or older, and usually no older than 100 years old, i.e., between the ages of about 50 and 100, e.g., 50, 55, 60, 65, 70, 75, 80, 85, 90, 95 or about 100 years old. In some cases a subject individual is suffering from cognitive impairment. In some cases a subject individual has not yet begun to show symptoms of cognitive impairment.

In some embodiments, the subject methods and compositions find use in slowing the progression of a central nervous system disorder (e.g., a brain disorder such as stroke). In other words, symptoms of the disorder in the individual will decline more slowly following treatment by the disclosed methods than prior to or in the absence of treatment by the disclosed methods. In some such instances, the subject methods of treatment include measuring the progression of symptoms of the disorder after treatment, and determining that progression is reduced. In some such instances, the determination is made by comparing to a reference, e.g., the rate of symptom decline in the individual prior to treatment, e.g., as determined by measuring symptoms at two or more time points prior to administration of a TREM1 inhibitory agent.

The subject methods and compositions also find use in stabilizing a a central nervous system disorder (e.g., a brain disorder such as stroke) of an individual, e.g., an individual suffering from a disorder or an individual at risk of suffering from a disorder. For example, the individual may demonstrate some symptoms of a disorder, and progression of symptoms observed prior to treatment with the disclosed methods will be halted following treatment by the disclosed methods. As another example, the individual may be at risk for developing a central nervous system disorder (e.g., a brain disorder such as stroke) (e.g., the individual may be aged 50 years old or older, or may have been diagnosed with an aging-associated disorder), and the symptoms (e.g., cognitive abilities) of the individual are substantially unchanged, i.e., no symptoms (e.g., no cognitive decline) can be detected, following treatment by the disclosed methods as compared to prior to treatment with the disclosed methods.

The subject methods and compositions also find use in reducing a central nervous system disorder (e.g., a brain disorder such as stroke) in an individual suffering from the disorder. In other words, symptom(s) (e.g., cognitive ability) are improved in the individual following treatment by the subject methods. For example, the symptom(s) in the individual are increased, e.g., by 2-fold or more, 5-fold or more, 10-fold or more, 15-fold or more, 20-fold or more, 30-fold or more, or 40-fold or more, including 50-fold or more, 60-fold or more, 70-fold or more, 80-fold or more, 90-fold or more, or 100-fold or more, following treatment by the subject methods relative to symptoms observed in the individual prior to treatment by the subject methods. In some instances, treatment by the subject methods and compositions are restorative (e.g., restore the cognitive ability) in the individual suffering from a central nervous system disorder (e.g., a brain disorder such as stroke), e.g., to their level when the individual was about 40 years old or less. In other words, the symptoms (e.g., cognitive impairment) are abrogated.

The subject methods and compositions find use in reducing, if not preventing, age-associated brain inflammation, neurodegeneration and cognitive decline.

In some embodiments, administration of a TREM1 inhibitory agent may be performed in conjunction with an active agent having activity suitable to treat a central nervous system disorder. For example, a number of active agents have been shown to have some efficacy in treating the cognitive symptoms of Alzheimer's disease (e.g., memory loss, confusion, and problems with thinking and reasoning), e.g., cholinesterase inhibitors (e.g., Donepezil, Rivastigmine, Galantamine, Tacrine), Memantine, and Vitamin E. As another example, a number of agents have been shown to have some efficacy in treating behavioral or psychiatric symptoms of Alzheimer's Disease, e.g., citalopram (Celexa), fluoxetine (Prozac), paroxeine (Paxil), sertraline (Zoloft), trazodone (Desyrel), lorazepam (Ativan), oxazepam (Serax), aripiprazole (Abilify), clozapine (Clozaril), haloperidol (Haldol), olanzapine (Zyprexa), quetiapine (Seroquel), risperidone (Risperdal), and ziprasidone (Geodon).

In some aspects of the subject methods, the method further comprises the step of measuring symptom(s) of a central nervous system disorder (e.g., cognition and/or synaptic plasticity) after treatment, e.g., using the methods described herein or known in the art, and determining that the rate of cognitive decline or loss of synaptic plasticity have been reduced and/or that cognitive ability or synaptic plasticity have improved in the individual. In some such instances, the determination is made by comparing the results of the cognition or synaptic plasticity test to the results of the test performed on the same individual at an earlier time, e.g., 2 weeks earlier, 1 month earlier, 2 months earlier, 3 months earlier, 6 months earlier, 1 year earlier, 2 years earlier, 5 years earlier, or 10 years earlier, or more.

In some embodiments, the subject methods further include diagnosing an individual as having symptom(s) of a central nervous system disorder (e.g., cognitive impairment), e.g., using the methods described herein or known in the art for measuring such symptom(s), prior to administering a subject TREM1 inhibitory agent. In some instances, the diagnosing will comprise measuring cognition and/or synaptic plasticity and comparing the results of the cognition or synaptic plasticity test to one or more references, e.g., a positive control and/or a negative control. For example, the reference may be the results of the test performed by one or more age-matched individuals that experience symptom(s) of a central nervous system disorder (i.e., positive controls) or that do not experience symptom(s) of a central nervous system disorder (i.e., negative controls). As another example, the reference may be the results of the test performed by the same individual at an earlier time, e.g., 2 weeks earlier, 1 month earlier, 2 months earlier, 3 months earlier, 6 months earlier, 1 year earlier, 2 years earlier, 5 years earlier, or 10 years earlier, or more.

In some embodiments, the subject methods further comprise diagnosing an individual as having a central nervous system disorder, e.g., stroke, Alzheimer's disease, Parkinson's disease, frontotemporal dementia, and the like (described elsewhere herein). Methods for diagnosing such disorders are well-known in the art, any of which may be used by the ordinarily skilled artisan in diagnosing the individual.

Compositions (e.g., a subject TREM1 inhibitory agent such as a blocking peptide) for the treatment of a brain disorder (e.g., stroke) can be administered by parenteral, topical, intravenous, intratumoral, oral, subcutaneous, intraarterial, intracranial, intraperitoneal, intranasal or intramuscular means. A typical route of administration is intravenous or intratumoral, although other routes can be equally effective. In some cases administration is systemic. In some cases administration is local. In some cases administration includes injection of a TREM1 inhibitory agent such as a blocking peptide.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of TREM1 inhibitory agents can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The TREM1 inhibitory agents, e.g., as described above, can be administered to a subject alone or in combination with an additional, i.e., second, active agent. As such, in some cases, the subject method further comprises administering to the subject at least one additional compound. Any convenient agents may be utilized, including compounds useful for treating the specific condition of interest. The terms “agent,” “compound,” and “drug” are used interchangeably herein. The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug with a pharmaceutical composition of the present disclosure means administration of the compound and second agent at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs and compounds of the present disclosure.

In some embodiments, the compounds (e.g., a subject compound and the at least one additional compound) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

Also within the scope of the invention are kits comprising a TREM1 inhibitory agent or formulations thereof and instructions for use. The kit can further contain a least one additional reagent. Kits typically include a label indicating the intended use of the contents of the kit. The term label includes any writing, or recorded material supplied on or with the kit, or which otherwise accompanies the kit. In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

### EMBODIMENTS

Non limiting embodiments of the present disclosure include the following.

1. A method of treating a central nervous system disorder in a subject, the method comprising

administering to the subject an effective amount of a TREM1 inhibitory agent sufficient to treat the subject for the central nervous system disorder.

2. The method of embodiment 1, wherein the TREM1 inhibitory agent inhibits TREM1 signaling activity.

3. The method of embodiment 1, wherein the TREM1 inhibitory agent reduces expression of TREM1.

4. The method of embodiment 3, wherein the TREM1 inhibitory agent is selected from the group consisting of: an antisense agent, an RNAi agent, and a genome editing agent.

5. The method of embodiment 1, wherein the TREM1 inhibitory agent is a TREM1 specific antibody or TREM1 binding fragment thereof.

6. The method of embodiment 1, wherein the TREM1 inhibitory agent is a TREM1 blocking peptide.

7. The method of embodiment 6, wherein the TREM1 blocking peptide comprises the amino acid sequence LQVTDSGLYRCVIYHPP (SEQ ID NO: 1) (LP17) or comprises the amino acid sequence LQEEDTGEYGCV (SEQ ID NO: 2) (LR12).

8. The method of embodiment 1, wherein the TREM1 inhibitory agent is a small molecule.

9. The method of embodiment 1, wherein TREM1 activity is reduced in the subject relative to the activity of TREM2.

10. The method of embodiment 1, wherein the TREM1 inhibitory agent is administered in a combination with at least one additional factor.

11. The method of embodiment 1, wherein the TREM1 activity is reduced in a cell in the subject relative to the activity of TREM2.

12. The method of embodiment 1, wherein the brain disorder is a condition exhibiting a maladaptive neuroinflammatory response.

13. The method of embodiment 12, wherein the brain disorder is selected from the group consisting of: stroke, head trauma, spinal cord injury, seizures, encephalitis, Alzheimer's disease, Parkinson's disease, fronto-temporal dementia, Parkinson's disease, amyotrophic lateral sclerosis, Huntington's disease, multiple sclerosis, pain, depression, PTSD, post-stroke inflammation, post-trauma inflammation, and chronic fatigue syndrome.

14. The method of embodiment 12, wherein the brain disorder is an acute brain disorder.

15. The method of embodiment 14, wherein the acute brain disorder is a stroke.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

We have determined that TREM1 is highly induced in microglia of the brain in acute and chronic brain disorders where the inflammatory response contributes significantly to brain injury and disease progression.

Specifically, in models of Alzheimer's disease (AD), we have determined that TREM1 is significantly upregulated in response to oligomeric Aβ peptides, which are the pathologic early instigators of injury in prodromal AD.

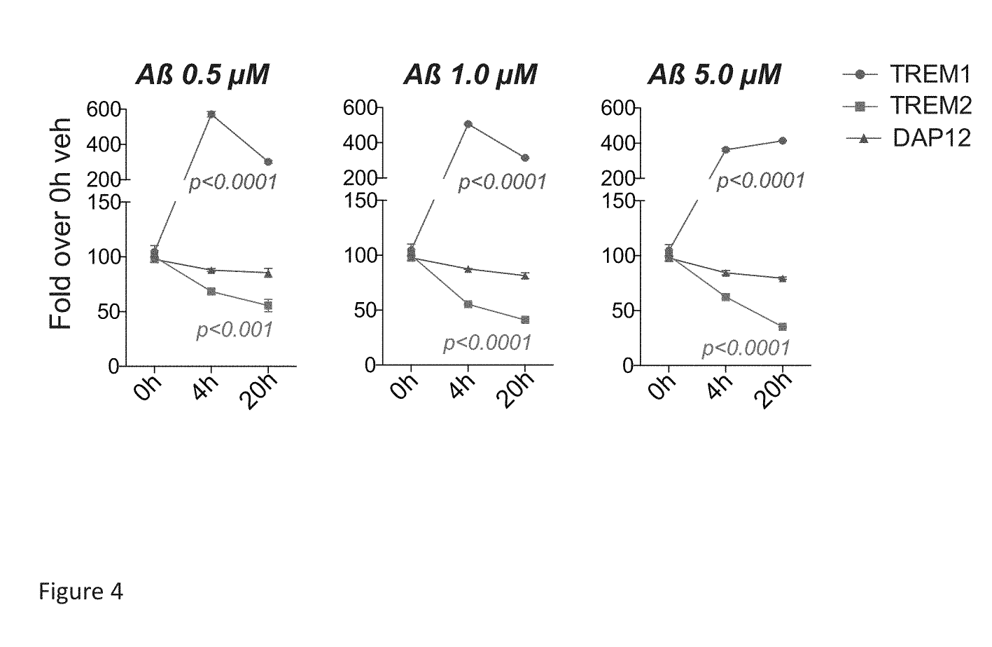

To validate the induction of TREM1, and to confirm a reciprocal decrease in expression of TREM2, we examined a dose-response and time course of TREM1, TREM2, and DAP12 expression in BV2 cells stimulated with Aβ₄₂ oligomers. As shown in FIG. 4, expression of TREM1 (red) increases and TREM2 (green) decreases in BV2 cells stimulated with Aβ₄₂ oligomers for 4h and 20h (one-way ANOVA; n=3 per group). DAP12 (blue) is minimally reduced (ANOVA p<0.01 at Aβ 1 μM; p<0.001 at Aβ 5 μM). Thus, a significant induction of TREM1 mRNA expression occurs after Aβ₄₂ stimulation, and this is associated with a converse and significant decline in TREM2 expression.

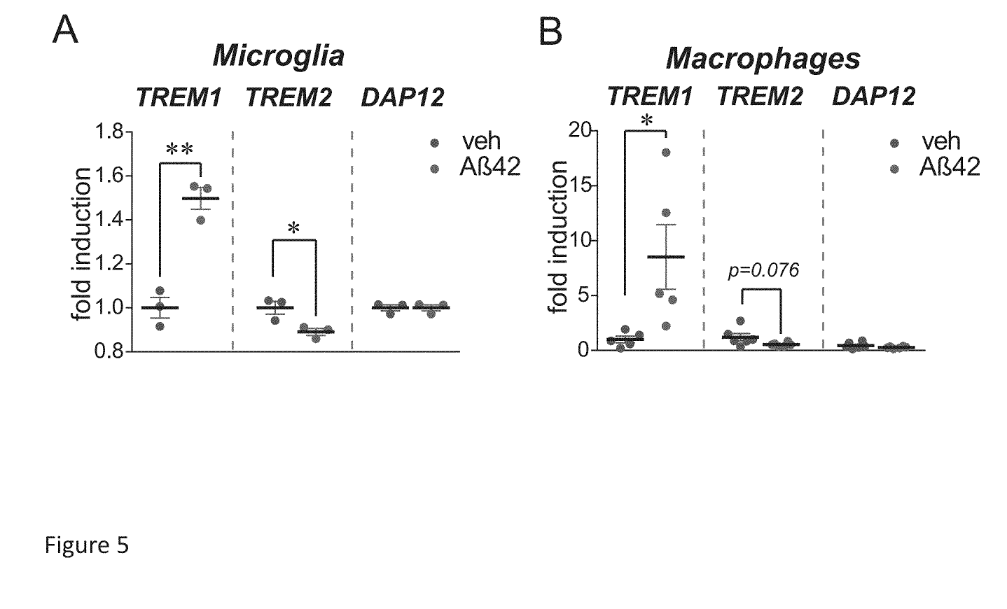

Postnatal primary microglia (postnatal, panel A; and 3 month adult peritoneal marcophages, panel B) were stimulated with oligomeric Aβ₄₂ (10 μM for 4 hours) and compared to vehicle stimulated cells. Transcriptional changes were assayed using Affimetrix GeneChip Mouse Gene 1.0 ST arrays. As shown in FIG. 5, TREM1 expression increased by 1.49 fold in Aβ₄₂ oligomer-stimulated microglia (p<0.01; panel A). Interestingly, TREM2 expression was simultaneously decreased with Aβ₄₂ oligomer stimulation (p<0.05), and DAP12 expression was unchanged. We then compared TREM1, TREM2, and DAP12 expression changes in another myeloid lineage cell type, and assayed effects of Aβ₄₂ oligomer stimulation on peritoneal macrophages (panel B). As in microglia, TREM1 was induced and TREM2 was reduced in response to Aβ₄₂ oligomers. Taken together, these data suggest that TREM1 and TREM2 are reciprocally regulated in the context of stimulation with immunogenic Aβ₄₂ oligomers. (In FIG. 5, *p<0.05, **p<0.01, unpaired t-test).

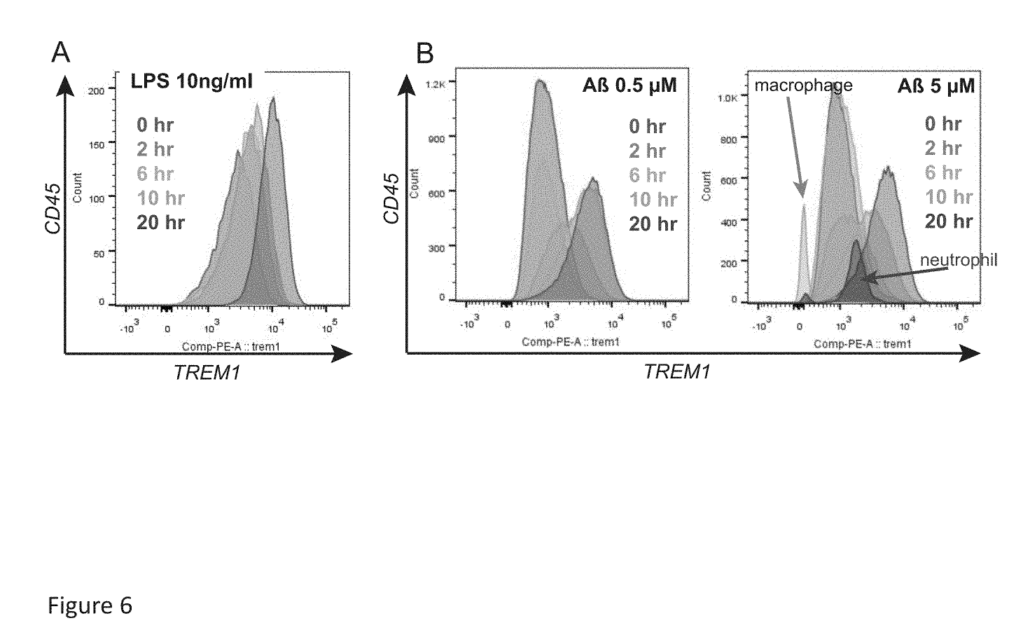

In addition, we have determined that TREM1 induction is similarly regulated in brain microglial cells in response to other immunogenic stimuli, for example lipopolysaccharide, a component of the bacterial cell wall, which is a classical innate immune stimulus. Mouse BV2 microglial cells were stimulated with LPS or Aβ₄₂ oligomers. Fluorescence-activated cell sorting (FACS) was carried out on cells harvested at 0 h, 2 h, 6 h, 10 h, and 20 h to detect TREM1 surface expression. As shown in FIG. 6, TREM1 expression increased with LPS (10 ng/ml; panel A) and with Aβ₄₂ 0.5 and 5.0 μM over 20 h (two B panels). Peripheral blood macrophages and neutrophils, which express low and high levels of TREM1, respectively, were run as negative and positive controls (B, rightmost panel).

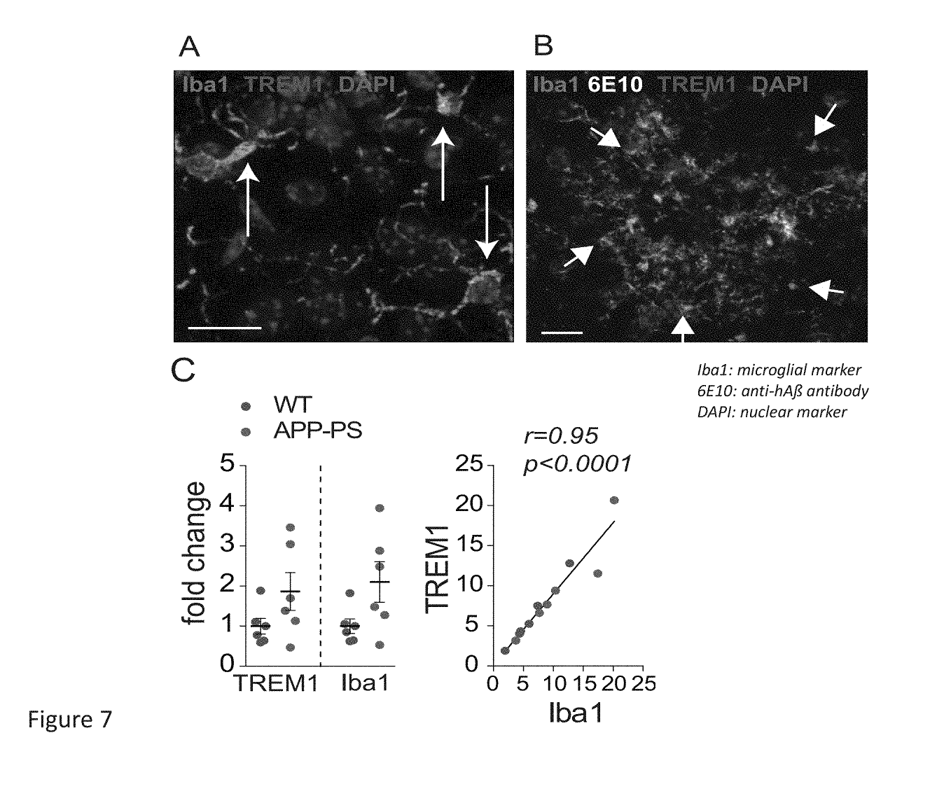

Confocal immuno-fluorescence was carried out on hippocampus from 9 mo APP-PS and WT littermates (mouse model of AD) and is shown in FIG. 7 panels A and B. TREM1 was undetectable in WT (not shown), but was increased in APP-PS microglia stained with Iba1 (immunostaining is shown in FIGS. 7A and 7B for rabbit anti-Iba1 (green), rat anti-mouse TREM1 (red), and mouse 6E10 to human Aβ (white)). Panel A: microglia in APP-PS1 dentate gyrus show colocalization of TREM1 and Iba1 (white arrows; scale bar=10 μM). Panel B: TREM1+/Iba1⁺ microglia occur at the periphery of amyloid plaques (white arrows; scale bar=10 μm). Panel C: Quantification of TREM1 and Iba1 intensities (n=6/group, 5-6 sections/mouse) and correlation (r=0.95; p<0.0001). Interestingly, in many amyloid plaques, TREM1⁺/Iba1⁺ cells appeared at the periphery the plaque, and not in more central regions of the plaque. TREM1 is upregulated in microglia and expression correlates highly significantly to microglial activation markers.

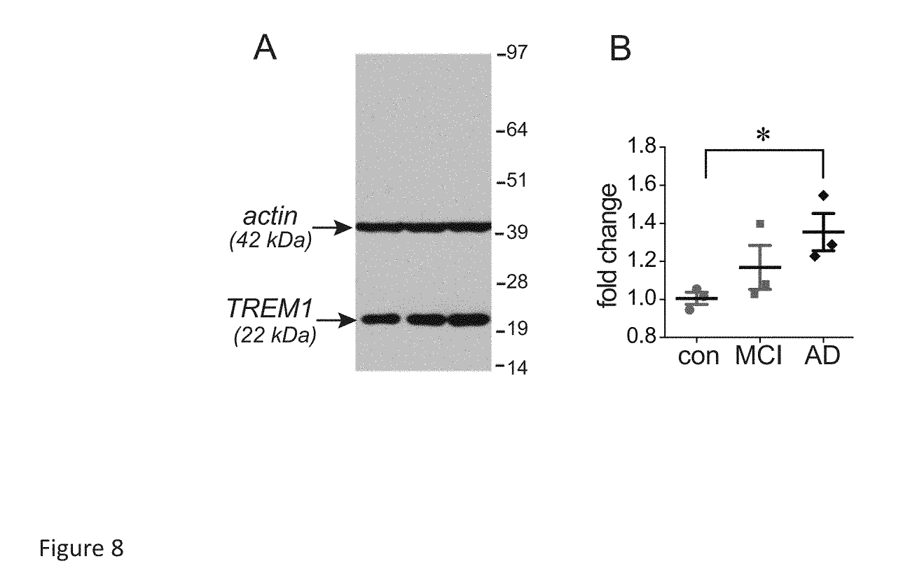

Recent evaluation of the TREM1 locus has identified an intronic variant in the promoter region of TREM1 associated with increased neuritic plaques and cognitive decline (Replogle J M, Chan G, White C C, Raj T, Winn P A, Evans D A, et al. A TREM1 variant alters the accumulation of Alzheimer-related amyloid pathology. Ann Neurol. 2015; 77(3):469-77. doi: 10.1002/ana.24337. PubMed PMID: 25545807) FIG. 8 shows TREM1 expression in human temporal neocortex. Panel A: Single band is detected by rabbit anti-human TREM1 antibody (1/1000; Abcam). Panel B: Data examining levels of TREM1 in post-mortem brain of control (con), mild cognitive impairment (MCI), and AD subjects demonstrates increased TREM1 expression in superior temporal neocortex with progression to AD (TREM1 quantification (*p<0.05)).

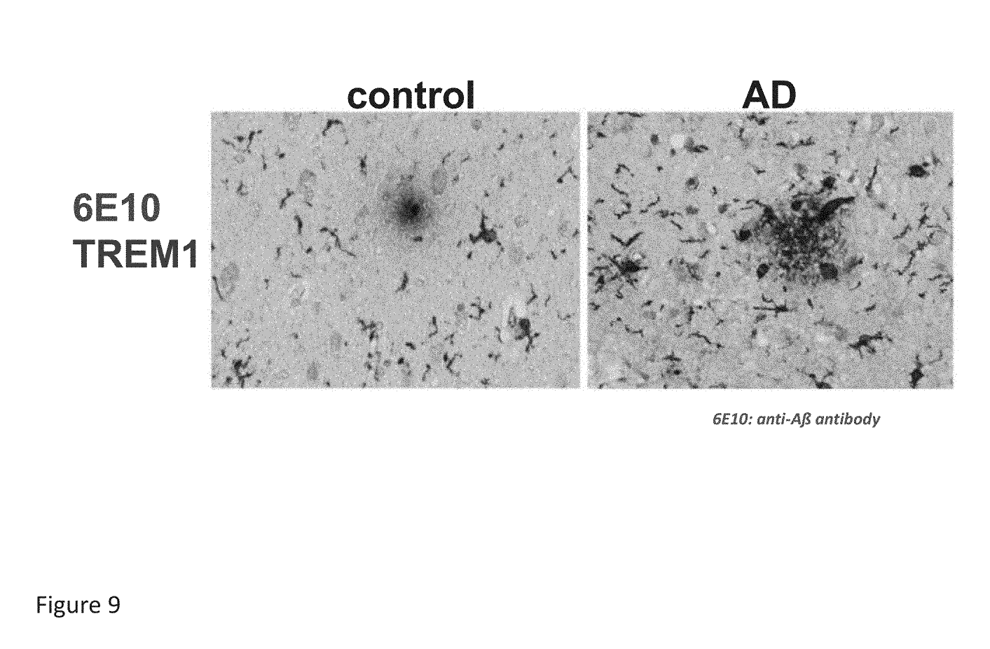

Control (Braak stage II/III) and AD (Braak stage V/VI) sections of superior temporal lobe were stained with anti-TREM1 antibody (brown) and 6E10 antibody against Aβ peptides (purple). As shown in FIG. 9, TREM1 was expressed in microglia in both control and AD tissues. Microglia were moderately stained with TREM1 in rare amyloid plaques in control brain (left), but in AD brain (right), TREM1 positive microglia appeared more numerous and morphologically activated around plaques.

We have also made the observation in brain microglial cells that TREM1 expression is inversely correlated with the expression of TREM2, its anti-inflammatory family member.

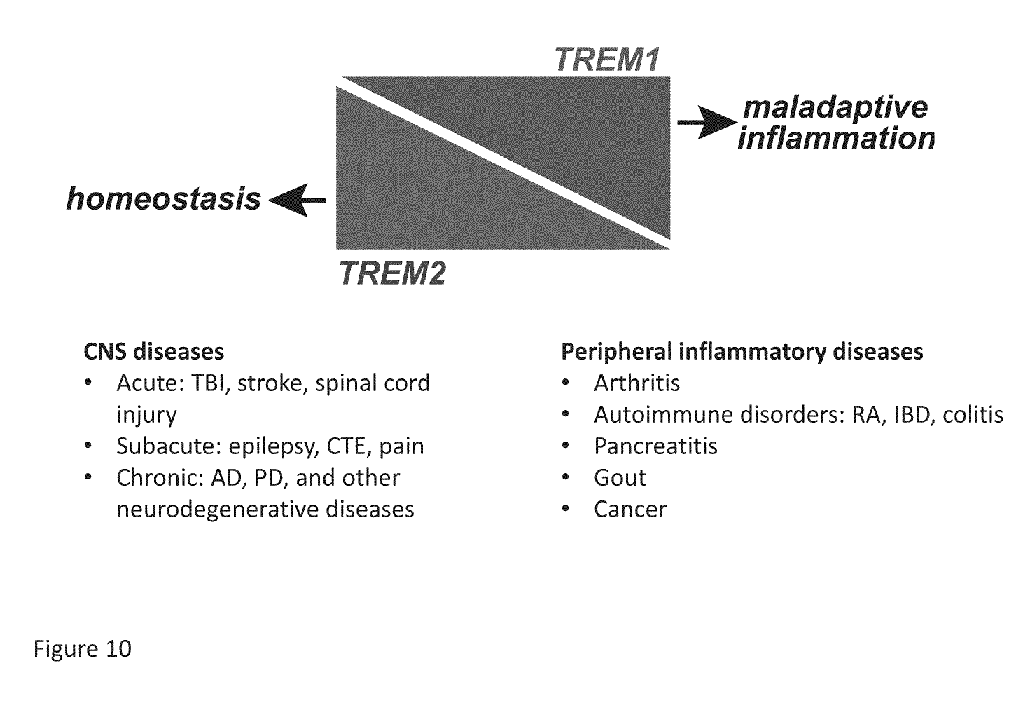

GWAS has identified variants in TREM2 to increase AD risk. FIG. 10. TREM1/TREM2 balance: relevance to CNS as well as peripheral diseases.

### Example 2

We have performed further experiments that demonstrate that TREM1 expression is highly induced in immune cells in the brain after an ischemic event, specifically after middle cerebral arterly occlusion (MCAo) followed by reperfusion (RP).

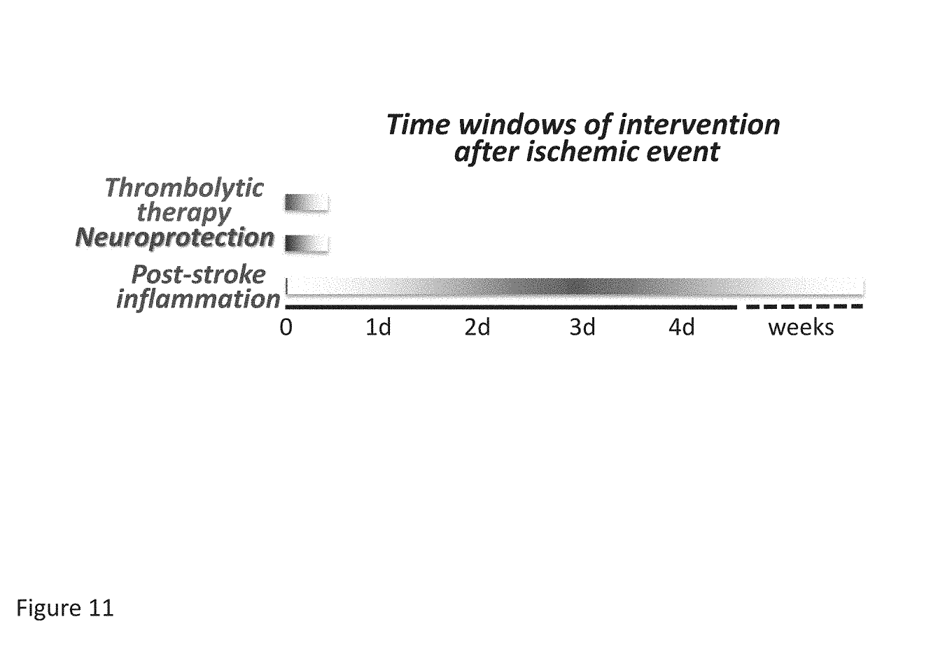

FIG. 11 shows a timeline of the window for intervention after an ischemic event (i.e., post-stroke). The window for a post-stroke inflammatory response is on the order of weeks as opposed to days for thrombolytic therapy and neuroprotection.

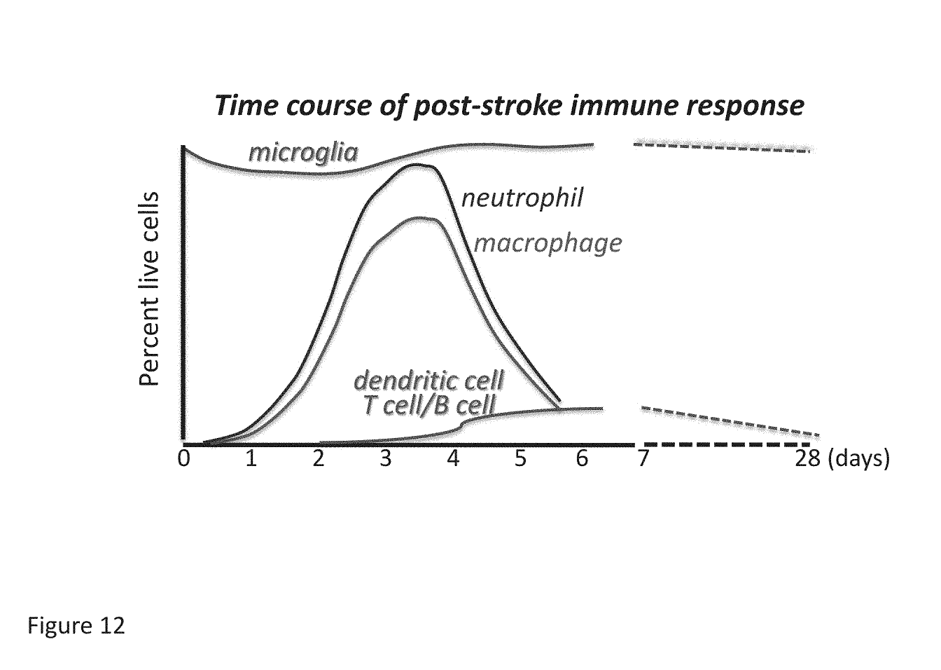

FIG. 12 shows a time course (in days) of the percent live cells that are immune cells in the brain (dendritic cells, T cells and B cells (brown line); macrophages (red line); and neutrophils (purple line)) and microglia (green line) after a stroke.

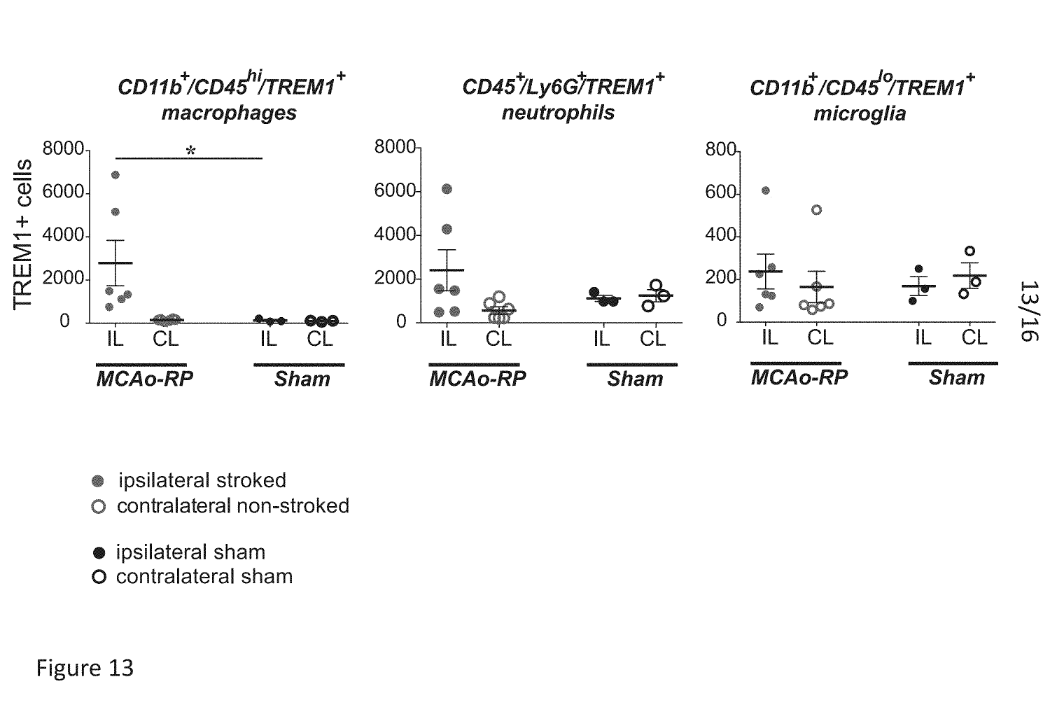

FIG. 13 shows FACS quantification plots of TREM1 positive macrophages (left panel), neutrophils (middle panel), and microglia (right panel) 2 days after MCAo-RP. As shown, TREM1 is increased in infiltrating myeloid cells in ischemic ipsilateral (IL) hemisphere as compared to contralateral (CL) and sham IL and CL hemispheres (n=3-6/group; *p<0.05 2-tailed t-test).

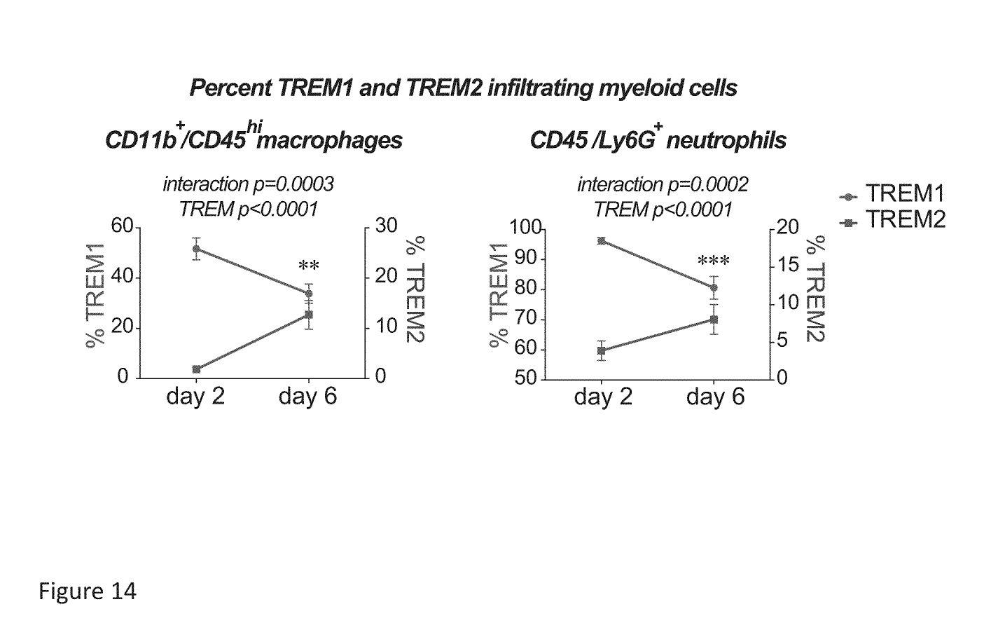

FIG. 14 shows that TREM1 and TREM 2 expression in infiltrating myeloid cells are inversely regulated at days 2 and 6 after MCAo-RP. Shown are FACS quantification of percentages of CD11b⁺/CD45^{hi} myeloid cells that are TREM1 (red) or TREM2 (green) positive, with macrophages (left panel) and neutrophils (right panel; n=5-6 per group). TREM1 is initially high (day 2) and then decreases (day 6) whereas TREM2 is initially low (day 2) and then increases (day 6). Post hoc Tukey **p<0.01 and ***p<0.001 for TREM1 day 2 vs day 6 for macrophages and neutrophils.

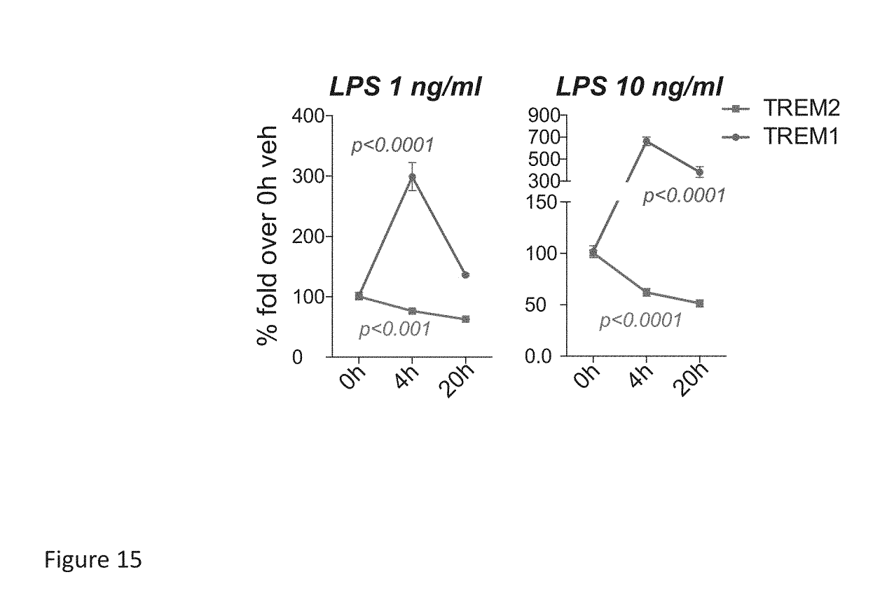

We found reciprocal expression of TREM1 and TREM2 in RAW mouse macrophage cell line in response to LPS. FIG. 15 shows that expression of TREM1 mRNA (red) increases and TREM2 mRNA (green) decreases in BV2 microglial cells and RAW macrophages stimulated with LPS for 4 h and 20 h, as assayed by qRT-PCR (Left panel: LPS 1 mg/ml; Right panel: LPS 10 ng/ml; one-way ANOVA; n=2-4 per group).

We found that a peptide decoy for TREM1 (LP17) reduces infiltration of macrophages after stroke. Panel A of FIG. 17 shows that neuroscores were improved 2 days after MCAo-RP in LP17-treated mice (n=15-18 per group; ANOVA: effect of LP17 p=0.008, post-hoc Bonferroni p<0.05 at day 1 for LP17 vs scrambled peptide). Panel B shows representative FACS plots of stroked hemispheres at 2 days after MCAo-RP. These plots show increased CD11b⁺/CD45^{lo} microglia and reduced CD11b⁺/CD45^{hi} macrophages with LP17 treatment. Panel C shows plots of the percent of total CD11b⁺/CD45⁺ myeloid cells at 2 days after MCAo-RP in ischemic ipsilateral (IL) and non-ischemic contralateral (CL) hemispheres that are microglia (left plot), macrophages (center plot), and neutrophils (right plot) (n=6-7 per group; post-hoc **p<0.01 for LP17 vs scrambled peptide).

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

What is claimed is:

1. A method of treating a central nervous system disorder in a subject, the method comprising
administering to the subject an effective amount of a TREM1 inhibitory agent sufficient to treat the subject for the central nervous system disorder.

2. The method of claim 1, wherein the TREM1 inhibitory agent inhibits TREM1 signaling activity.

3. The method of claim 1, wherein the TREM1 inhibitory agent reduces expression of TREM1.

4. The method of claim 3, wherein the TREM1 inhibitory agent is selected from the group consisting of: an antisense agent, an RNAi agent, and a genome editing agent.

5. The method of claim 1, wherein the TREM1 inhibitory agent is a TREM1 specific antibody or TREM1 binding fragment thereof.

6. The method of claim 1, wherein the TREM1 inhibitory agent is a TREM1 blocking peptide.

7. The method of claim 6, wherein the TREM1 blocking peptide comprises the amino acid sequence LQVTDSGLYRCVIYHPP (SEQ ID NO: 1) (LP17) or comprises the amino acid sequence LQEEDTGEYGCV (SEQ ID NO: 2) (LR12).

8. The method of claim 1, wherein the TREM1 inhibitory agent is a small molecule.

9. The method of claim 1, wherein TREM1 activity is reduced in the subject relative to the activity of TREM2.

10. The method of claim 1, wherein the TREM1 inhibitory agent is administered in a combination with at least one additional factor.

11. The method of claim 1, wherein the TREM1 activity is reduced in a cell in the subject relative to the activity of TREM2.

12. The method of claim 1, wherein the brain disorder is a condition exhibiting a maladaptive neuroinflammatory response.

13. The method of claim 12, wherein the brain disorder is selected from the group consisting of: stroke, head trauma, spinal cord injury, seizures, encephalitis, Alzheimer's disease, Parkinson's disease, fronto-temporal dementia, Parkinson's disease, amyotrophic lateral sclerosis, Huntington's disease, multiple sclerosis, pain, depression, PTSD, post-stroke inflammation, post-trauma inflammation, and chronic fatigue syndrome.

14. The method of claim 12, wherein the brain disorder is an acute brain disorder.

15. The method of claim 14, wherein the acute brain disorder is a stroke.
